(19) United States
(12) Patent Application Publication
(10) Pub. No.: US 2011/0288504 A1
(43) Pub. Date: Nov. 24, 2011

(54) THERMOREGULATION PACKS AND RELATED METHODS

(75) Inventor: Fernando Migliassi, Miami, FL (US)

(73) Assignee: DANIALE LLC, Miami, FL (US)

(21) Appl. No.: 13/113,956

(22) Filed: May 23, 2011

Related U.S. Application Data
(60) Provisional application No. 61/347,357, filed on May 21, 2010.
(60) Provisional application No. 61/385,288, filed on Sep. 22, 2010.

(51) Int. Cl. A61F 7/02; A61M 35/00

(52) U.S. Cl. 604/289; 607/108

(57) ABSTRACT

In one embodiment, a thermoregulation device to be worn by a user comprises a body having one or more receiving portions, the body having an axis extending about its height, wherein the receiving portions extend along the axis and each removably receive and hold in position a thermoregulation material; and at least one strap coupled to at least one portion of the body and extend about a portion of the user when worn by the user. The at least one strap assists in holding the body tightly against a spine of the user when worn such that the axis is substantially aligned with the spine of the user. And the portion and the receiving portions hold the thermoregulation material in position along the axis such that the thermoregulation material is held in proximity and adjacent to the spine of the user when worn by the user.

This application claims the benefit of U.S. Provisional Application No. 61/347,357, filed May 21, 2010, and claims the benefit of U.S. Provisional Application No. 61/385,288, filed Sep. 22, 2010, both of which are incorporated in their entirety herein by reference.

### FIELD OF THE INVENTION

The present invention relates generally to body temperature regulation. The present invention more particularly, though not exclusively, relates to body temperature regulation of athletes during athletic activities.

### BACKGROUND OF THE INVENTION

The ability to sense and regulate body temperature (“thermoregulation”) is a key feature of human survival. The human body has thermoregulatory mechanisms that play key roles in maintaining physiological homeostasis during both rest and physical exercise. Such homeostasis is often challenged by physical exertion which causes a substantial increase in metabolic heat production. In fact, during intense prolonged physical exertion, such as during endurance races, body temperature can increase from about 37° C. at rest, to more than 42° C.

When the body temperature rises too high, human organs can fail, and the central nervous system can be temporarily or permanently impaired. In fact, a temperature deviation of as little as ±3.5° C. from the resting temperature of 37° C. can result in physiological impairments and fatality. For instance, heat stroke is a life-threatening heat disorder that is caused by a complete collapse of a body's thermoregulatory mechanisms, and characterized by: increased internal body temperature to a value exceeding 40° C.; cessation of sweating; hot and dry skin; rapid pulse and respiration; hypertension (high blood pressure); confusion; and eventually unconsciousness.

Heat stroke does not just occur under excessively hot external temperatures, but can even occur in moderate environmental temperatures (70° F. with a humidity of 30%) as well. If left untreated, heat stroke will progress to a coma, and death will quickly follow. Common treatments for heat stroke victims involve a rapid cooling of the body in a bath of ice water or wrapping the athlete in cold, wet sheets and fanning the body to remove excess heat.

In humans, body temperature comprises the temperatures of the core and shell. Core temperature (Tc) refers to the temperatures inside of the abdominal, thoracic and cranial cavities, and shell temperature (Ts) refers to the temperatures of the skin, subcutaneous tissue and muscles. Tc is regulated by the brain, and is typically about 36.8° C. during rest. The Ts is influenced more by skin blood flow and environmental conditions such as surrounding air temperature and wind. For example, exposure to cold ambient temperature decreases Ts, but Tc may remain relatively constant. Humans are unique in that they are able to maintain constant temperature utilizing the endothermic Tc, regulated by the brain, in combination with the ectothermic Ts, which is influenced by external environment.

The human body is able to capitalize on the dual-thermic properties of thermoregulation by making the Ts slave to Tc. When the body core temperature is too hot, the blood flow to the skin is increased, resulting in an elevated Ts and a corresponding increase in heat dissipation to the environment. When the body core temperature is too low, the blood flow to the skin is decreased, resulting in a decrease in Ts and a corresponding decrease in heat dissipation to the environment.

Heat transfer between the body and the external environment occurs through the processes of conduction, convection, radiation and evaporation. Heat transfer through convection, conduction and radiation is bidirectional, and the amount of heat transferred between the skin surface and the environment is driven largely by the temperature gradient between the skin and the surrounding environment. Heat is transferred from the environment to the skin if the ambient temperature is warmer than Ts, and heat is transferred from the skin to the environment if the Ts is warmer than the ambient temperature.

Unlike convection, conduction and radiation, heat dissipation through evaporation is uni-directional; heat is transferred only from the skin surface to the external environment. Evaporative heat loss occurs when sweat changes from a liquid to gaseous state. During physical exercise, the majority of heat is dissipated through evaporative heat loss, making it the primary means of cooling the body. Therefore, the ability to sweat is very important for thermoregulation and the sustenance of exercise over long periods. However, the ability for sweat to be evaporated is inversely related to the amount of water vapor in the air. For instance, a high relative humidity slows evaporative heat loss whereas evaporative heat loss is increased when the relative humidity in the air is low. As a result, exercising under a warm and humid condition may cause the body to lose fluid through sweat, yet will not provide the athlete with sufficient cooling effects to maintain the Tc at a safe level.

Athletes that are engaged in strenuous exercises must have a mechanism for facilitating heat loss from their bodies. Failure to dissipate heat will result in dangerous medical conditions, and perhaps lead to heat stroke and even death. Because that athlete may engage in strenuous exercise through different climate conditions, it is necessary to develop a mechanism for cooling the Ts sufficiently to have a cooling effect on the Tc as well, without being dependent upon ever-changing climate conditions.

In light of the dangers outlined above, it is advantageous to provide an apparatus to assist an athlete in regulating their body temperatures during strenuous exercise. It is likewise advantageous to provide a mechanism to facilitate the cooling and heating of a body shell, thereby affecting the core temperature of an athlete. It is also advantageous to provide a device that is easy to use, relatively easy to manufacture, and comparatively cost effective.

### SUMMARY OF THE INVENTION

Several embodiments provide thermoregulating devices and related methods of regulating thermal characteristics of users.

In one embodiment, a thermoregulation device to be worn by a user comprises a body portion having one or more receiving portions, the body portion having an axis extending about a height of the body portion, wherein the one or more receiving portions extend along the axis and each of the one or more receiving portions is configured to removably receive and hold in position a thermoregulation material; and at least one strap coupled to at least one portion of the body portion and configured to extend about a portion of the user when worn by the user. The at least one strap is configured to assist in holding the body portion tightly against a spine of the user when worn such that the axis is substantially aligned with the spine of the user. And the body portion and the one or more receiving portions are configured to hold the thermoregulation material in position along the axis such that the thermoregulation material is held in proximity and adjacent to the spine of the user when worn by the user.

In another embodiment, a thermoregulation device to be worn by a user, the thermoregulation device comprises: a body portion having one or more receiving portions, the body portion having an axis extending about a height of the body portion, wherein the one or more receiving portions extend along the axis and each of the one or more receiving portions is configured to removably receive and hold in position a thermoregulation material; and a coupling mechanism coupled to a first portion of the body portion and configured to removably couple the first portion of the body portion to a portion of a garment also worn by the user when the thermoregulation device is worn, wherein the coupling mechanism is configured to assist in holding the thermoregulation material in position along the axis such that the thermoregulation material is held in proximity and adjacent to the spine of the user when worn by the user.

In a further embodiment, a method of regulating a thermal characteristic of a user during physical exertion, the method comprises: inserting a thermoregulation material into one or more receiving portions of a body portion of a thermoregulation device, wherein the body portion has an axis extending about a height of the body portion, wherein the one or more receiving portions extend along the axis and each of the one or more receiving portions is configured to removably receive and hold in position the thermoregulation material along the axis; and positioning the body portion proximate to a back of the user such that the thermoregulation material is held in proximity and adjacent to a spine of the user; and using at least one strap coupled to at least one portion of the body portion to assist in holding the body portion tightly against the spine of the user such that the axis is substantially aligned with the spine of the user, wherein the at least one strap extends about a portion of the user.

### BRIEF DESCRIPTION OF THE DRAWINGS

The features of embodiments of the invention, as well as the embodiments themselves, both as to its structure and its operation, will be best understood from the accompanying drawings, taken in conjunction with the accompanying description, in which similar reference characters refer to similar parts.

FIG. 1 is a plan view of the thermoregulation pack for users having a breathable backing pad formed with an upper pouch and a lower pouch designed to easily accept and retain reusable or single use heat/cooling packs, a collar clip for attachment to a user's shirt collar, two upper shoulder strap attachment points and two corresponding lower shoulder strap attachment points in accordance with some embodiments.

FIG. 2 is an opposite side plan view showing the thermoregulation pack for users with two installed heat/cooling packs (shown in phantom) in accordance with some embodiments.

FIG. 3 is a top perspective view of a thermoregulation pack for users including a collar clip for attachment to a user's shirt collar, and two upper shoulder strap attachment points, two lower shoulder strap attachment points and with the pouches each containing a heat/cooling pack in accordance with one embodiment.

FIG. 4 is a top perspective view of the thermoregulation pack of FIG. 3 showing two heat/cooling packs partially inserted into the upper and lower pouches in accordance with one embodiment.

FIG. 5 is a view of an upper edge of the backing pad and including a collar clip for attachment to a user's shirt collar, and two upper shoulder strap attachment points in accordance with one embodiment.

FIG. 6 is a top view showing a collar clip prior to attachment to the collar of a user's shirt in accordance with one embodiment.

FIG. 7 is a top view showing the collar clip of FIG. 6 attached to the collar of a user's shirt to provide additional vertical support to the thermoregulation pack as it is being worn by a user during exercise in accordance with one embodiment.

FIG. 8 is a side view of a collar clip prior to attachment to the collar of a user's shirt in accordance with one embodiment.

FIG. 9 is a side view of the collar clip of FIG. 8 as attached to the collar of a user's shirt in accordance with one embodiment.

FIG. 10 is a top plan view of the thermoregulation pack of one embodiment showing the left and right shoulder straps extending from the two upper shoulder strap attachment points and two corresponding lower shoulder strap attachment points.

FIG. 11 is a view of the thermoregulation pack of one embodiment as worn by a user showing the left and right shoulder straps extending from the two upper shoulder strap attachment points and two corresponding lower shoulder strap attachment points to position the heat/cooling packs on the user's back adjacent the spine to thereby provide maximum contribution to either heating or cooling the user's shell and core temperatures.

FIG. 12 is a diagrammatic representation of an alternative embodiment of the thermoregulation pack for users having a single pouch formed to accept a single heat/cooling pack, and having integrated shoulder straps extending from the backing pad.

FIG. 13 is a view of the alternative embodiment of the thermoregulation pack showing the left and right integrated shoulder straps.

FIG. 14 is a view of another embodiment of a thermoregulation pack including integrated shoulder straps, a collar clip and a horizontal chest strap.

FIG. 15 is a view of another embodiment of a thermoregulation pack having a coupling mechanism, integrated adjustable shoulder straps, and an adjustable chest strap in accordance with some embodiments.

FIG. 16 is a view of the thermoregulation pack of FIG. 15 with the chest strap attached.

FIG. 17 is a back view of the thermoregulation pack of FIGS. 15 and 16 illustrating two receiving portions.

FIG. 18 is an illustration of one embodiment of a flap of the receiving portion of the thermoregulation pack of FIGS. 15 and 16.

FIG. 19 shows views of several variations of thermoregulation materials embodied as packs having multiple sections in multiple sizes in accordance with some embodiments.

Corresponding reference characters and/or other identifiers indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Embodiments of the present invention are directed to devices intended to facilitate the cooling or heating of a user, such as an athlete. In one embodiment, a thermoregulation pack for users includes a breathable backing pad, or a body portion, that is formed with an upper receiving portion (e.g., upper pouch) and a lower receiving portion (e.g., lower pouch) each designed to accept a thermoregulation material (e.g., reusable heat/cooling packs). In one embodiment, an upper edge of the body portion includes a coupling mechanism (e.g., a collar clip) for removable attachment to a user's garment (e.g., shirt collar), and two strap attachment mechanisms (e.g., two upper shoulder strap attachment points). In one embodiment, a lower edge of the body portion includes two strap attachment mechanisms (e.g., two lower shoulder strap attachment points). A strap (e.g., a shoulder strap) is removably attachable between a lower attachment mechanism and an upper attachment mechanism so that a user can wear the thermoregulation pack for users by putting the straps over his or her arms, and the thermoregulation pack for users will press against the user's back adjacent the spine. Accordingly, in one embodiment, the body portion and the receiving portions are configured to hold the thermoregulation material in position such that the thermoregulation material is held in proximity and adjacent to the spine of the user when worn by the user.

In an alternative embodiment, the thermoregulation pack for users is formed to have a single elongated receiving portion (e.g., pouch) designed to accept an elongated thermoregulation material (e.g., a single elongated heat/cooling pack).

In another embodiment, the thermoregulation pack may be formed with integrated shoulder straps that extend outwards from the upper and lower edges of the body portion and are adjustable to fit the user. In an alternative embodiment, the shoulder straps may be attached to the thermoregulation pack by shoulder strap attachment mechanisms, and may be removable from the thermoregulation pack. In a further embodiment, the thermoregulation pack may include an adjustable, horizontal strap that crosses the chest and/or abdomen and adjoins either the integrated or removable shoulder straps. In another embodiment, the thermoregulation pack may be formed without straps.

In some embodiments, the body portion or backing pad is manufactured from synthetic material having different qualities on each side. More specifically, in one example, the side of the fabric that touches the body is engineered to draw/absorb moisture from the body (user), while the side of the fabric opposite the body (user) is designed to wick and expel the moisture and dissipate it away from the fabric. This moisture transfer mechanism assists in the cooling function of the thermoregulation pack for users in some embodiments.

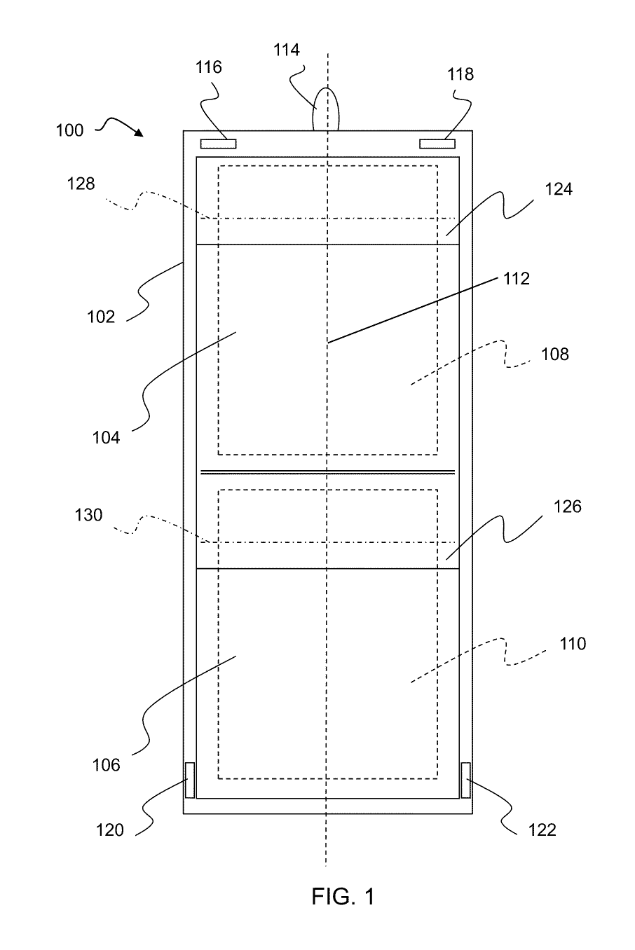

Referring to FIG. 1, a plan view of the thermoregulation pack 100 or sleeve (also referred to as a thermoregulation device) for users, such as athletes, having a breathable backing pad or body portion 102 according to one embodiment is shown. The body portion 102 is formed with an upper pouch 104 (generically referred to as an upper receiving portion) and a lower pouch 106 (generically referred to as a lower receiving portion) designed to easily accept and retain reusable heat/cooling packs 108 and 110 (each generically referred to as a thermoregulation material). The heat/cooling packs contain a heating/cooling substance. For example, each pouch has an opening to removably receive and contain a heat/cooling pack within a volume of the pouch. In an alternative embodiment, the backing pad or body portion may be formed to have a single elongated receiving portion (e.g., pouch) designed to accept a single elongated thermoregulation material (e.g., heat/cooling pack).

The thermoregulation pack 100 for users of several embodiments may be utilized to warm or cool a user. For instance, while exercising in cold environments (high winds, freezing temperatures, etc.), it may be advantageous to provide a user with a heat source to assist in maintaining Ts and Tc.

Reusable thermoregulation materials, such as heating and cooling packs, are known in the art. In some cases, the heat packs are exothermic pouches that contain a chemical combination that, once activated, produces heat through chemical reactions. In other cases, the heat packs are gel-based and require that the pouch be heated prior to use.

Reusable cooling packs are typically specially formulated gel-matrix thermal packs that are placed in a freezer prior to use. The cooling packs will typically remain flexible even when frozen, and provide a malleable cooling pack for adaptation to nearly any surface contour. These formable cooling packs will maintain a cooled temperature for long periods, and are well suited for incorporation into some embodiments of the present invention.

As an alternative to the gel-based cooling packs, endothermic cooling packs may be used as the thermoregulation material. These packs contain a chemical combination that, once activated, consumes heat through chemical reactions. Also, the pouches may be filled with bags of ice (another example of a thermoregulation material) to provide the cooling source. Also, the ice may be placed directly into the pouch such that when the ice begins to melt, the cold water passes through the pouch material and onto the user thereby increasing the amount of evaporative cooling that can take place.

In various embodiments, cooling and/or heating packs can come in different sizes. For example, a small cooling pack may be 11 cm wide×19 cm long with 250 grams of gel, a medium cooling pack may be 12.5 cm wide×20.5 cm long with 300 grams of gel, and a large cooling pack may be 15 cm wide×23 cm long with 360 grams of gel. For the different sizes of cooling packs, the corresponding sleeve or thermoregulation pack may be sized accordingly. For example, in a two pouch embodiment, such as illustrated in FIG. 1, a small thermoregulation pack for use with small cooling packs may be about 11.5 cm wide×40.5 cm long, a medium pack for use with medium cooling packs may be about 13 cm wide×43.5 cm long, and a large pack for use with large cooling packs may be about 15.5 cm wide×48.5 cm long. In a second example, an extra-small cooling pack may be 7.4 cm wide×12.7 cm long with 100 grams of gel, a small cooling pack may be 9 cm wide×15.5 cm long with 150 grams of gel, a medium cooling pack may be 11 cm wide×19 cm long with 250 grams of gel, and a large cooling pack may be 13 cm wide×22.5 cm long with 350 grams of gel, and an extra-large cooling pack may be 15.4 cm wide×26.7 cm long with 400 grams of gel. In a second example of a two pouch embodiment, an extra-small thermoregulation pack for use with extra-small cooling packs may be about 7.8 cm wide×23.8 cm long, a small thermoregulation pack for use with small cooling packs may be about 9.4 cm wide×29.4 cm long, a medium pack for use with medium cooling packs may be about 11.4 cm wide×36.4 cm long, and a large pack for use with large cooling packs may be about 13.4 cm wide×43.4 cm long, and an extra-large thermoregulation pack for use with extra-large cooling packs may be about 15.8 cm wide×51.8 cm long. It is noted that in this second embodiment, the length of the packs is slightly less than the length of two corresponding sized cooling packs, and that this is ok due to the stretch of the fabric of the thermoregulation pack in this embodiment. That is, in some embodiments, one or more of the thermoregulation packs described herein may be at least partially made from a material or fabric that is stretchable. In some embodiments, the weight of the small thermoregulation pack is approximately 22 grams. The different thermoregulation pack sizes may be provided to accommodate users of different sizes and ages, and may also be provided to provide a different length of time of cooling or heating, e.g., depending on how long an athlete will be participating in an activity. While cooling packs are used in the above examples, it is understood that heating packs may be interchangeably used in these size examples. In further embodiments, the mass of cooling or heating material within a given size cooling/heating pack may be varied to vary the length of time that cooling/heating will be provided, e.g., a given size cooling/heating pack may be made in different thicknesses. For example, the more material contained within a given cooling/heating pack, the longer length of time cooling/heating will be provided. The design of the amount of thermoregulation material within a given pack can be chosen to provide the desired cooling/heating effect for the desired amount of time and at a desired weight. In some embodiments, while it is desired to provide sufficient cooling, simply providing more cooling material will add more weight and so the designer balances the weight and cooling/heating effect with the use in mind. For example, in cycling, it is desired not to add significant weight to the pack as worn by the rider. Additionally, in some embodiments, the thermoregulation material is divided into multiple sections, such as described in relation to FIG. 19 below.

Also illustrated in FIG. 1 is an axis 112 of the thermoregulation pack 100 that is generally vertical, and in some embodiments, corresponds to the vertical line of the user's spine. In the illustrated embodiment, the pouches extend along the axis 112 relative to each other. Again, each of the pouches (one embodiment of receiving portions) removably receives and holds in position a thermoregulation material. In some embodiments, the body portion 102 and the pouches 104 and 106 hold the heating/cooling packs 108 and 110 in position along the axis 112 such that the heating/cooling packs 108 and 110 are held in proximity and adjacent to the spine of the user when worn by the user.

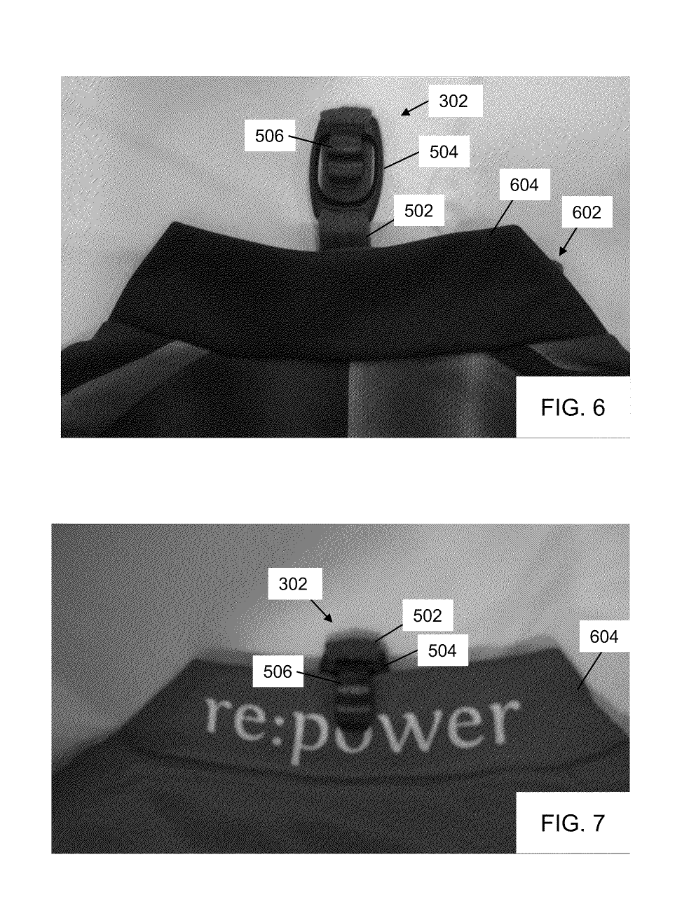

FIG. 1 also shows one embodiment of a collar clip 114 extending from the upper edge of the backing pad for attachment to a user's shirt collar. The collar clip 114 is one embodiment of a coupling mechanism which is generally configured to removably couple a portion of the body portion 102 to a portion of a garment also worn by the user when the thermoregulation device 100 is worn. In this embodiment, the collar clip 114 is configured to removably couple an upper portion of the body portion 102 to a collar of the user's shirt, bib short, etc. to help support the weight of the heating/cooling packs 108 and 110. Thus, the collar clip 114 generally assists in holding the thermoregulation material in position along the axis 112 such that the thermoregulation material is held in proximity and adjacent to the spine of the user when worn by the user. The collar clip 114 is shown below in conjunction with the embodiments of FIGS. 6-9.

In some embodiments, the pack 100 may be used with one or more straps, such as shoulder straps and/or horizontal straps (such as a chest strap and/or an abdominal strap). Such strap/s would extend about a portion of the user when worn by the user and assist in holding the body portion 102 tightly against a spine of the user when worn such that the axis 112 is substantially aligned with the spine of the user. To facilitate the attachment and detachment of straps, FIG. 1 illustrates two upper shoulder strap attachment mechanisms 116 and 118 and two corresponding lower shoulder strap attachment mechanisms 120 and 122. A shoulder strap (not shown in FIG. 1) extends vertically between the upper and lower attachment mechanisms.

FIG. 1 also illustrates that in one embodiment of a receiving portion, the upper pouch 104 includes an upper flap 124 and the lower pouch 106 includes a lower flap 126. In some embodiments, the flaps 124 and 126 cover an opening of the pouch (e.g., cover an upper pouch edge 128 and a lower pouch edge 130) and help retain the thermoregulation material therein.

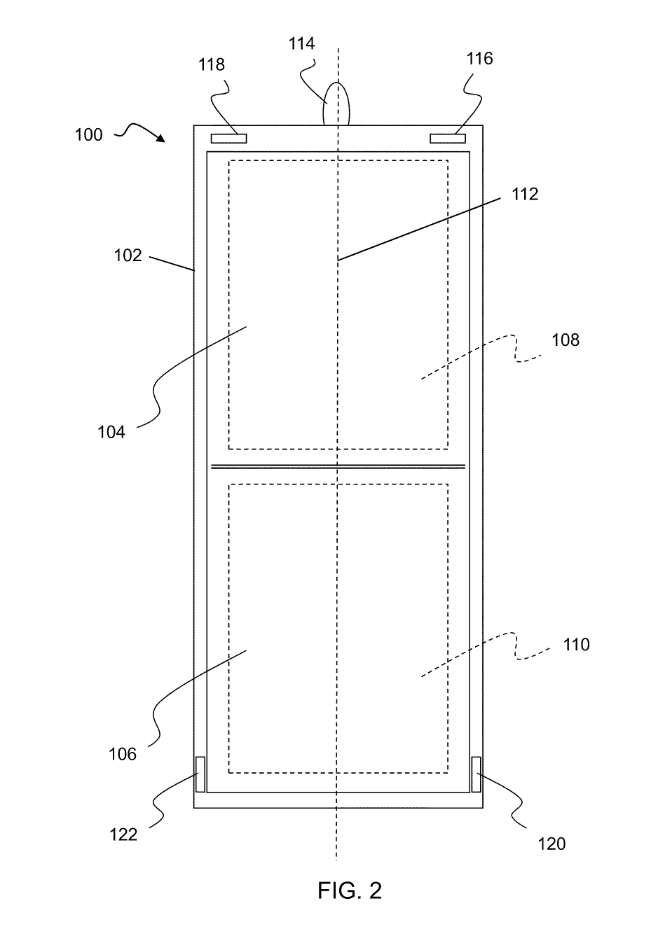

Referring now to FIG. 2, an opposite side plan view shows the thermoregulation pack for users with two installed heat/cooling packs (shown in phantom). This view shows the relative smoothness of the backing pad that, in use, will contact the user's back and locate adjacent the spine. Proper positioning along the spine assures that the pouches containing the heat/cooling packs are fully against the user's skin surface in order to maximize the effect this embodiment has on a user's Ts and corresponding Tc. In other words, it is generally desired to align the axis 112 of the pack 100 with the user's spine when the pack is worn by the user so that the thermoregulation material is held in proximity and adjacent to the spine of the user when worn by the user.

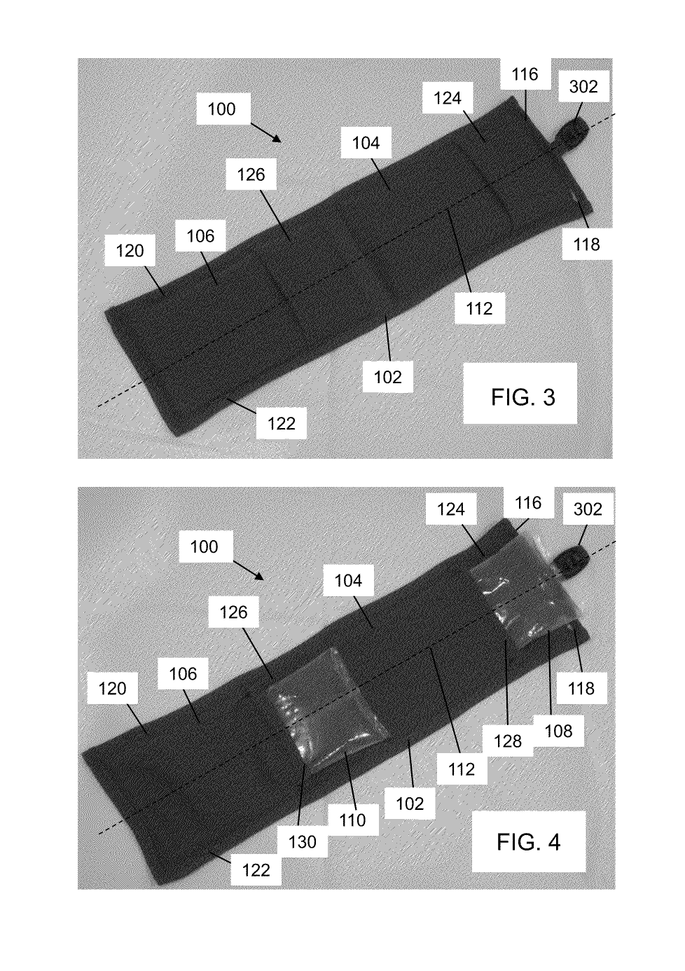

FIGS. 3, 4, and 5 are top views of the thermoregulation pack 100 for users of some embodiments. FIG. 3 illustrates the heat/cooling packs fully inserted into the upper pouch 104 and the lower pouch 106 with the flaps 124 and 126 in position. FIG. 4 show the two heat/cooling packs 108 and 110 partially inserted into the upper pouch 104 and the lower pouch 106. FIGS. 3-5 also illustrate a collar clip 302 in accordance with one embodiment for attachment to a user's shirt collar, two upper shoulder strap attachment mechanisms 116 and 118.

FIGS. 3-5 also illustrate an upper edge of the backing pad or body portion 102 and includes the collar clip 302 for attachment to a user's shirt collar according to one embodiment. This upper edge, in one embodiment, is formed from backing pad material and folded to provide a durable location from which to position the two upper shoulder strap attachment mechanisms 116 and 118. The upper pouch 104 is shown to contain a heat/cooling pack as is known in the art.

FIGS. 3-5, also illustrate that each pouch is formed with an over-flap 124 and 126 which serves to retain the heat/cooling pack within a volume of the pouch 104/106 during exercise. The over-flap 124/126 can be equipped with a fastener (e.g., see FIG. 18) to further assure that the pack will remain within the pouch. For instance, a hook-and-loop fastener, such as VELCRO, can be used to maintain the flap 124/126 in a closed position. Other fasteners known in the art, including but not limited to, zippers, buttons, and snaps, can be used to securely close over-flap to the pouch thereby capturing the pack.

FIGS. 3 and 4 also illustrate the lower portion of the thermoregulation pack of one embodiment showing the two lower shoulder strap attachment mechanisms 120 and 122. The strap attachment mechanisms may be formed as slots or alternatives known in the art may be used without departing from the present invention. For instance, loops may be sewn into the backing pad, or eyelets may be formed in the backing pad.

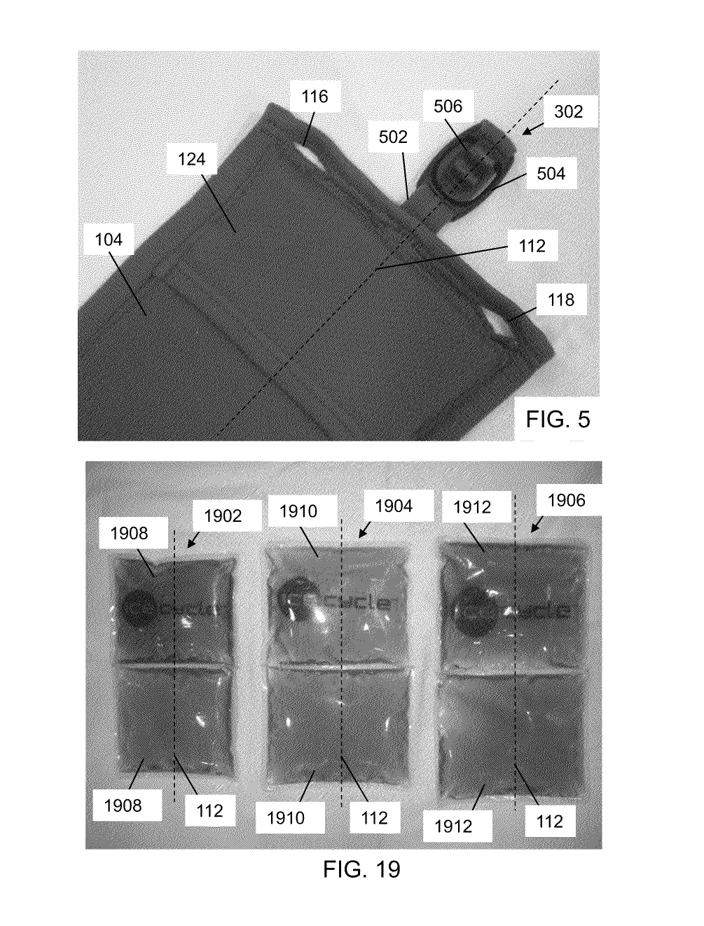

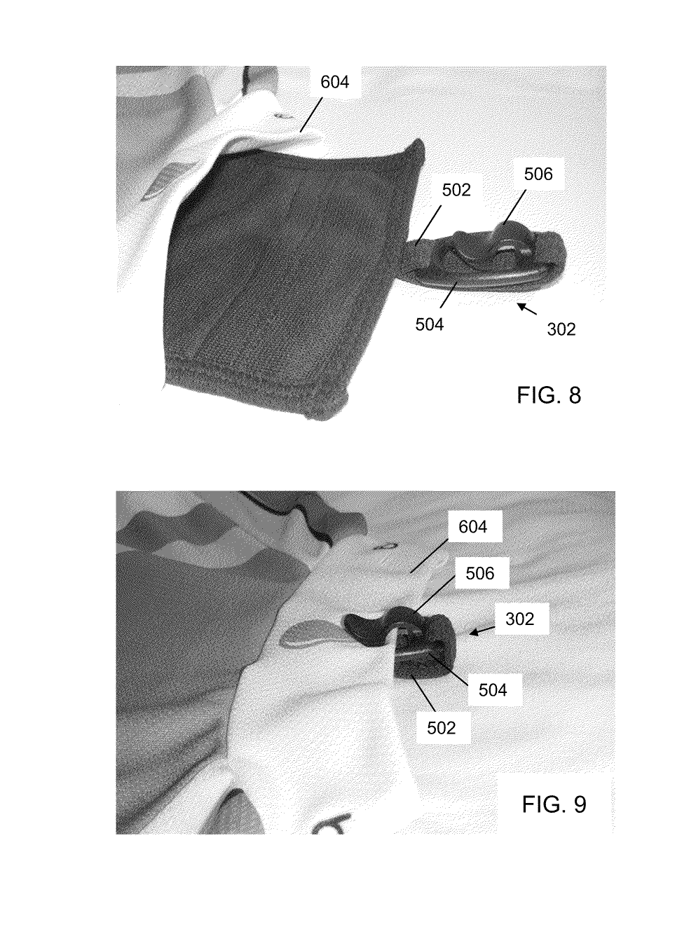

FIG. 5 also illustrates that the collar clip 302 includes a neck portion 502, a frame portion 504 and a clip portion 506, all of which are clearly illustrated in FIGS. 6-9. Generally, the collar clip is one embodiment of a coupling mechanism that is configured to removably couple a portion of the body portion 102 to a portion of a garment also worn by the user when the thermoregulation device is worn. Such a coupling mechanism is also configured to assist in holding the thermoregulation material in position along the axis 112 such that the thermoregulation material is held in proximity and adjacent to the spine of the user when worn by the user. FIGS. 6 and 8, show plan and side views, respectively, of the collar clip 302 of some embodiments as it would attach to the collar 604 of a user's shirt 602, and FIGS. 7 and 9 show plan and side view, respectively, of the collar clip 302 attached to the collar 604 of a user's shirt 602 to provide additional vertical support to embodiments of the thermoregulation pack as it is being worn by a user during exercise. In the illustrated embodiment, the neck portion 502 couples the frame portion 504 to a portion of the body portion 102. In one form, the neck portion 502 is a fabric strap that threads through a portion of the frame portion 504. The clip portion 506 extends from the frame portion 504 such that the collar 604 slides between the frame portion 504 and the clip portion 506 and is generally held by spring tension. In one form, the frame portion 504 and the clip portion 506 are made of a rigid but slightly flexible, plastic material. The portions 504 and 506 function similar to a paper clip holding paper. When the clip 302 is to be removed from the collar 604, the user simply slides the clip vertically upwards to remove the collar from between the frame portion 504 and the clip portion 506. It is noted that the clip portion 506 is integral with the frame portion 504 at its upper end so that the clip helps to support the weight of the pack 100 and help hold the thermoregulation material in position along the axis 112 such that the thermoregulation material is held in proximity and adjacent to the spine of the user when worn by the user. During exercise, it is important in some embodiments that the thermoregulation sleeve or pack maintains contact with the user in order to optimize the cooling or heating effects of the device. By fastening the upper edge of the backing pad to the user's collar, gravity pulls the pad or body portion 102 into contact with the user's back. As can be seen, the collar clip is configured such that the thermoregulation pack of this embodiment is worn under a user's shirt, i.e., the clip portion 506 faces away from the user's neck.

In some embodiments, the use of the collar clip 302 provides the ability to secure the thermoregulation sleeve or pack to the user's shirt collar (or collar of a bib short or other garment) thereby preventing the thermoregulation pack from sliding down the user's back. Further, some forms of the collar clip provide the user a non-cumbersome and non-intrusive way to secure the thermoregulation pack to the user while, at the same time, allowing the user the ability to easily remove the clip and the thermoregulation pack without the need for removing the user's shirt or jersey. This will allow the user to replace a thawed thermoregulation pack and replace it with a new, frozen thermoregulation pack. This is advantageous, for example, for athletes such as cyclists who may require the need to efficiently exchange a thermoregulation pack while staying on their bicycle during a race because they cannot afford the loss of time it would require to pull over to the side of the road, remove their shirt, remove a thawed thermoregulation pack, put on a new thermoregulation pack, put on their shirt, and then re-join the race which has continued throughout the entire replacement process.

In some embodiments, the collar clip may be utilized with a thermoregulation pack that has either integrated or removable shoulder straps, and also with a thermoregulation pack that does not have shoulder straps. In one example, with a thermoregulation pack without shoulder straps, the collar clip helps maintain the thermoregulation pack in a secure position on the user's body. In another example, for a thermoregulation pack with or without shoulder straps, the thermoregulation pack may be held in place by the compression of the user's clothing pressing the thermoregulation pack against the user's skin, with the collar clip assisting in maintaining the thermoregulation pack in a secure position on the user's body. As another example, for the thermoregulation pack with integrated shoulder straps, the collar clip may be used to ensure additional security and placement on the user's back.

In one embodiment, the collar clip may utilize a plastic piece (e.g., frame portion 504 and clip portion 506) that is secured to the main body of the thermoregulation pack by a fabric attachment (e.g., neck portion 502), thus, joining and integrating the clip with the thermoregulation pack.

It is understood that the term collar clip is used for embodiments of coupling mechanisms in which a top portion of the thermoregulation pack is clipped to a collar of the user's shirt, jacket, jersey, bib short or other garment. In accordance with other embodiments, the collar clip may be more generically referred to as a “coupling mechanism” that mechanically and removably couples at least a portion of the thermoregulation sleeve or pack to at least a portion of the user's garment, such as a shirt, jersey, jacket, bib short, shorts, or other outerwear. Such a coupling mechanism assists in holding the thermoregulation material in position along the axis 112 such that the thermoregulation material is held in proximity and adjacent to the spine of the user when worn by the user. The coupling mechanism may have one or more of the benefits of the collar clips described thus far. In some embodiments, the coupling mechanism is configured such that the thermoregulation pack is worn under the garment, while in other embodiments, the coupling mechanism is configured such that the thermoregulation pack is worn over the garment. In some embodiments, the coupling mechanism is configured to allow for a temporary and removable coupling to the garment, such as provided by embodiments of the collar clip.

In some embodiments, the coupling mechanism may take a variety of forms. For example, in some embodiments, the coupling mechanism take the form of magnets, zippers, buttons, clips (with or without a spring tension mechanism), fabric hook and fastener (e.g., VELCRO) and/or other hook-type fasteners. For example, in one embodiment, a magnet is integrated into or fixed to a portion of the backing pad 102 and a corresponding and attracting separate magnet is positioned on a surface of the user's shirt opposite that magnet of the backing pad. Variations of the coupling mechanism may have one or more of the benefits of the collar clips described thus far and/or other benefits not specifically associated with the collar clip or other coupling mechanisms.

In alternate embodiments, the coupling mechanism may be attached to other portions of the user's garments, including, but not limited to, other portions of a shirt, pants, shorts, and/or bib shorts (in common use with cyclists). Further, in some embodiments, the thermoregulation pack may include more than one coupling mechanism (such as multiple clips for fastening to a user's clothing). Additionally, while the collar clip is illustrated as being fixed to a top or upper portion of the thermoregulation pack, in some embodiments, the coupling mechanism may be fixed to one or more other portions of the pack. For example, instead of or in addition to a coupling mechanism at the top portion of the pack, one or more coupling mechanisms may be fixed to a central portion, a side portion and/or a bottom portion of the pack. In some embodiments, the coupling mechanism is configured such that the user's clothing does not need to be modified in order to be worn in conjunction with the thermoregulation pack. In other embodiments, the user's clothing may need to be modified, such as by adding a hole for a button.

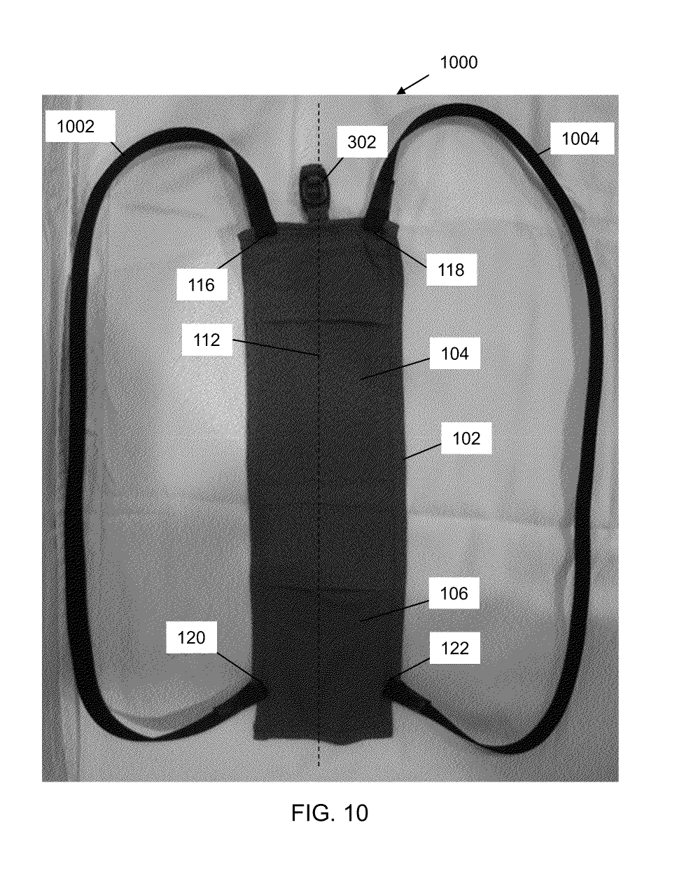

FIG. 10 is a top plan view of one embodiment of a thermoregulation pack 1000 showing a left shoulder strap 1002 and a right shoulder strap 1004 extending from the two upper shoulder strap attachment mechanisms 116, 118 and two corresponding lower shoulder strap attachment mechanisms 120, 122. In some embodiments, to facilitate easy attachment and detachment, end portions of each strap 1002 and 1004 extend through the respective strap attachment mechanisms 116, 118, 120, 122 (e.g., slots), fold back and removably couple back to another portion of the strap, e.g., using a fabric hook and loop fastener.

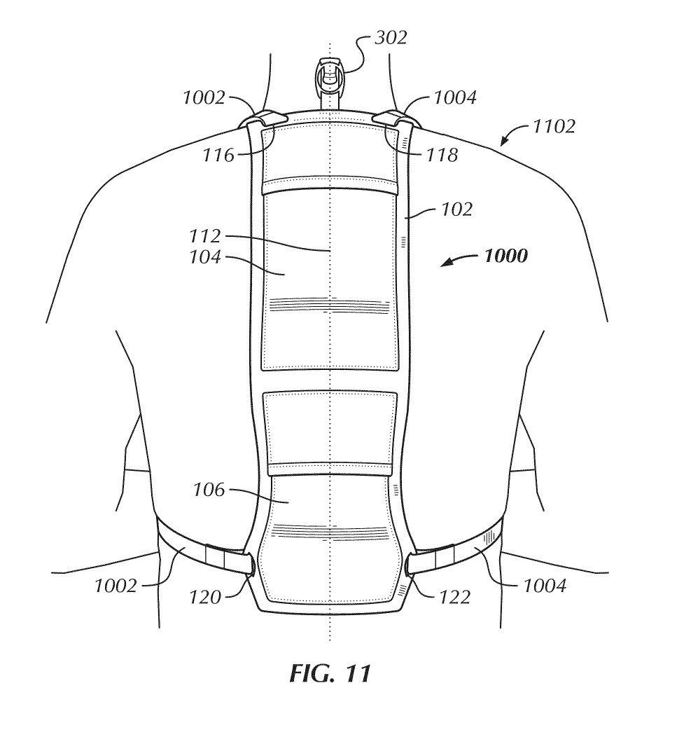

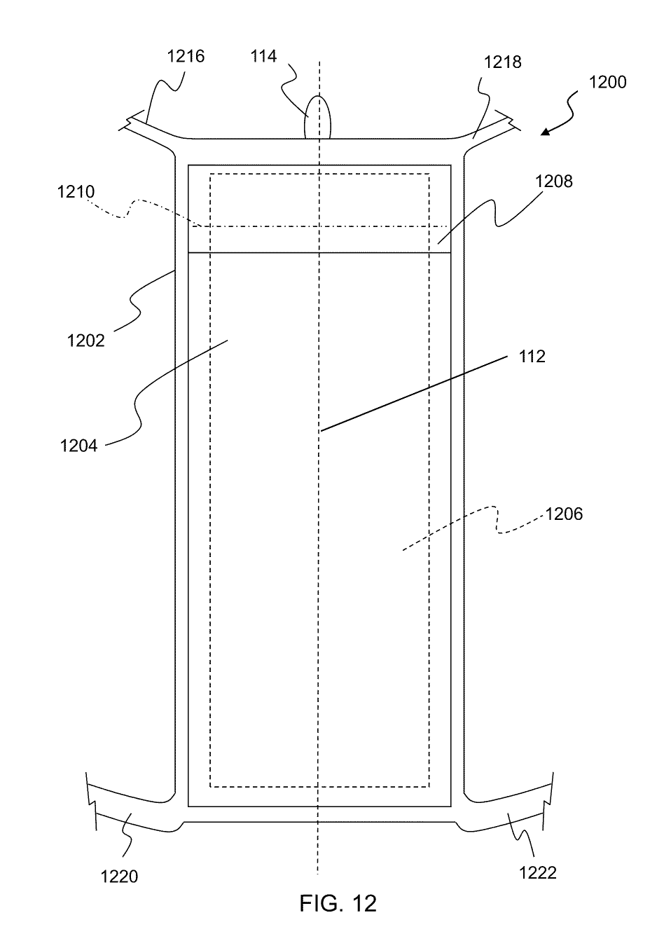

As seen in the embodiment of FIG. 11, the thermoregulation pack 1000 is worn by a user 1102 showing the left and right shoulder straps 1002, 1004 extending from the two upper shoulder strap attachment mechanisms 116, 118 and two corresponding lower shoulder strap attachment mechanisms 120, 122. When worn, the thermoregulation is in position with the heat/cooling packs substantially aligned to the axis 112 such that the heat/cooling packs are positions proximate to and adjacent the user's spine to thereby provide maximum contribution to either heating or cooling the user's core temperature. As can be seen in the embodiment of FIG. 11, the thermoregulation pack 1000 tightly fits against the user's body and aligns the cooling/heating packs against the user's spine.

FIG. 11 illustrates the thermoregulation pack 1000 being worn directly against the skin of the user 1102 to optimize cooling and/or heating. Accordingly, it is seen that due to the tight fitting nature of the pack 1000, the user may wear a shirt or jersey over the pack 1000, such as a fitted shirt or jersey. In some embodiments, the thermoregulation pack 1000 may be worn over the user's shirt. It is noted that the collar clip 302 illustrated in FIG. 11 is oriented such that it can clip or removably couple to a collar of a shirt or bib short worn over the pack 1000. In the event the pack were to be configured to be worn over the user's shirt, in some embodiments, the collar clip is reversed relative to that shown in FIG. 11, i.e., the clip portion of the collar clip would face inward. Referring now to FIG. 12, a diagrammatic representation of an alternative embodiment of the thermoregulation pack 1200 for users is shown and includes a body portion 1202 having a single receiving portion (e.g., pouch 1204 with flap 1208 having a pouch edge 1210) formed to accept a thermoregulation material (e.g., a single heat/cooling pack 1206). In this embodiment, a single heat/cooling pack is provided and based on the form factor, a larger volume of cooling or heating pack may be provided. Moreover, since the elongated pouch 1204 is not divided in the middle (as in the embodiment shown in FIG. 1), the pack may be thicker thereby holding a larger volume of cooled or warmed material.

The embodiment of FIG. 12 includes integrated or integral shoulder straps extending from the backing pad. That is, shown are upper shoulder straps 1216 and 1218 and lower should straps 1220 and 1222. These integrated straps eliminate the need for any clips or other forms of attachment between the strap and the backing pad or body portion 1202. In one embodiment, straps 1216 and 1220 are one integrated strap. Likewise, straps 1218 and 1222 are one integrated strap. In another embodiment, straps 1216, 1218, 1220 and 1222 are each separate straps that can removably connect together. For example, strap 1216 would removably attach or connect to strap 1220, and strap 1218 would removably attach or connect to strap 1222. The attachment may be made in any number of ways, e.g., a fabric hook and loop fastener, a ladder lock fastener, a loop lock fastener, a buckle fastener, and so on. In some embodiments, the removable connection is adjustable so that the proper fit or tightness can be selected by the user. Additionally, in some embodiments one or more of the straps (or any of the straps described herein) may be made of an elastic material such that the strap can be stretched when put on, but it returns to tightly hold the body portion to the back of the user. FIG. 12 also illustrates the collar clip 114 as one embodiment of a coupling mechanism.

Another embodiment of the integrated strap design shown in FIG. 12 may include two pouches instead of one, e.g., an upper pouch and a lower pouch as in FIG. 1. In this embodiment, the two heating/cooling packs in the two pouches will evenly divide the amount of heating/cooling material used, thus, limiting the ability of the soft gel (when used for heating or as the gel thaws when used for cooling) to slide to the bottom of the pack. In this manner, the thermoregulation material is distributed along the axis 112 and remains substantially distributed, e.g., as cooling material thaws. One skilled in the art would recognize that more than two pouches may be used; however, in some embodiments, the multiple pouches are positioned along the axis 112 relative to each other.

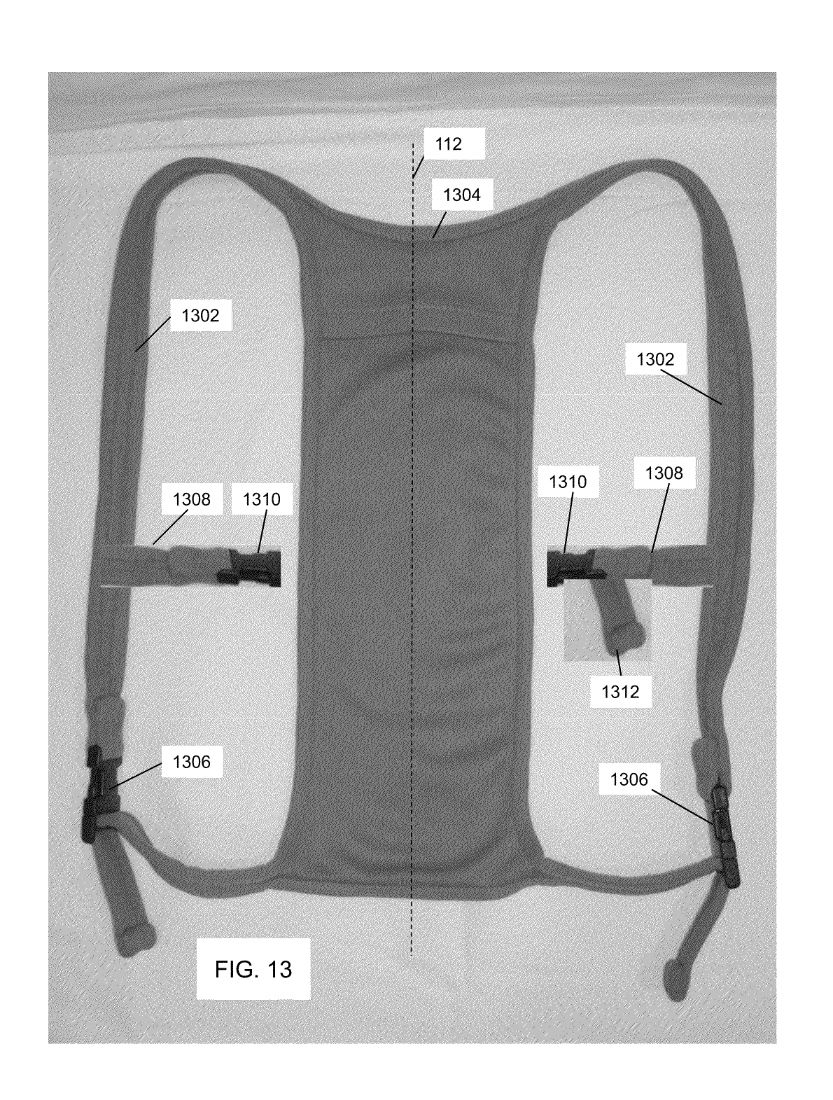

It is noted that the straps 1216, 1218, 1220 and 1222 attach at the body portion at an angle so as to assist in conforming to the shoulders of a user to allow the pack to fit tightly to the user's body. The same angular attachment is also shown in the embodiments of FIGS. 13-17.

FIG. 13 is a view of another embodiment of a thermoregulation pack 1300 showing the left and right shoulder straps 1302 extending from two upper portions and two lower portions of the body portion 1304. As shown, the shoulder straps 1302 and backing pad or body portion 1304 are integrated into one piece and the straps are adjustable using shoulder strap connector and adjustment 1306. In some embodiments, the straps 1302 and body portion 1304 may be made of the same performance fabric. In other embodiments, they are made of different materials. FIG. 13 depicts a design that is made to hold one long thermoregulation material (e.g., cooling/heating pack) instead of two shorter packs as in previous designs. In other embodiments, the thermoregulation pack may include two or more pouches instead of one. Further, the thermoregulation pack may include one or more collar clips or other coupling mechanisms, as described above.

In further embodiments, the thermoregulation pack includes horizontal straps 1308 that cross the chest and adjoin both shoulder straps 1302. The horizontal straps 1308 are connected using horizontal strap connector 1310. The horizontal straps are designed to allow the user the ability to further adjust, using the horizontal strap adjustment 1312, and secure the pack to the user's upper torso. The horizontal straps 1308 may be made of the same performance fabric as the rest of the thermoregulation pack and the two shoulder straps.

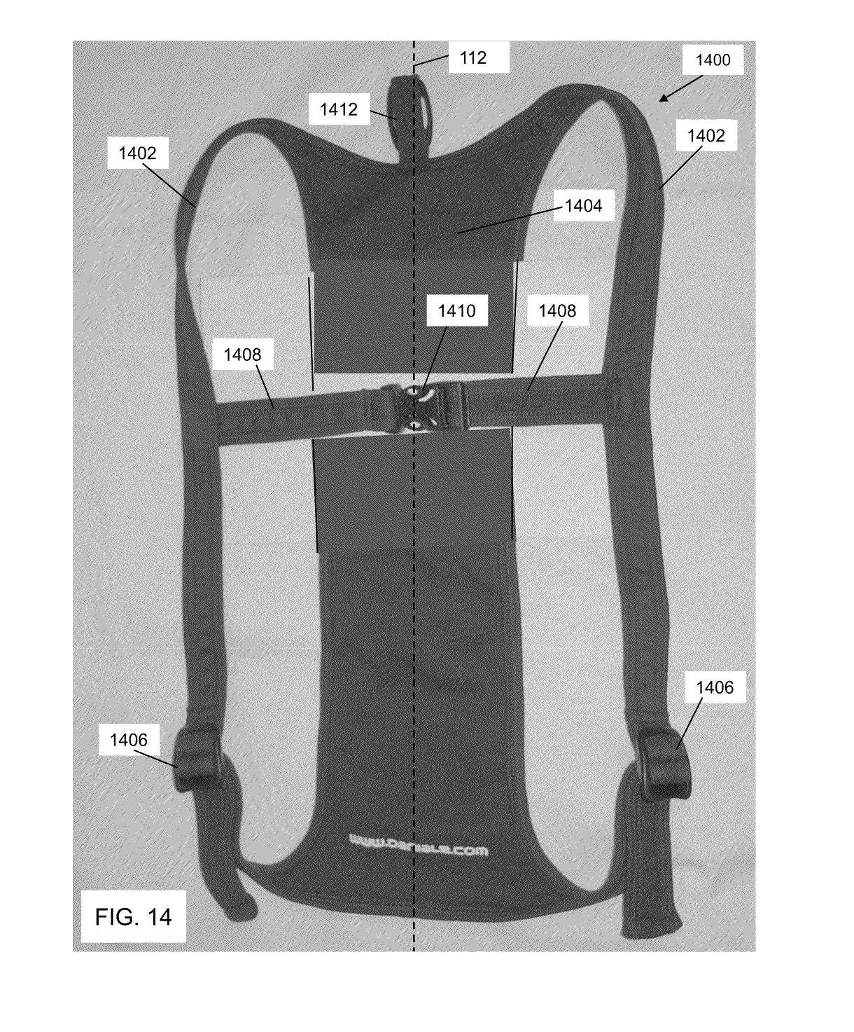

Referring to FIG. 14, shown is another embodiment of a thermoregulation pack 1400 including a backing pad or body portion 1404 with integrated adjustable shoulder straps 1402, a collar clip 1412 and a horizontal strap 1408 in which the connectors 1410 are adjoined, for example, to implement a chest strap. The shoulder straps 1402 are adjustable with shoulder strap connector and adjustment 1406.

In some embodiments, a coupling mechanism, such as a collar clip, will not be sufficient in order to keep the thermoregulation pack secured to the user's body. For example, a user who is a firefighter or a long distance runner who is constantly standing and moving will need more stability for the thermoregulation pack than the collar clip because gravity will naturally pull the pack down and thus pull on the user's collar or other portion of the garment. It will be in these types of circumstances that the thermoregulation pack with either removable or integrated straps will be most useful. In embodiments having adjustable straps, the tightness of the fit of the pack against the user's body can be adjusted.

In some embodiments, the illustrated collar clips and buckles described above are commercially available from ITW Nexus of Des Plaines, Ill. Further, in some embodiments, the dimensions of the various pieces may be as follows: the collar clip is about 2.39 cm wide×3.69 cm long; the ladderloc buckles that may be used on the shoulder straps measure about 2.9 cm wide×3.15 cm long; and side release buckles that may be used on the chest strap are about 2.6 cm wide×4.78 cm long.

In some embodiments, the breathable backing pad or body portion is a material with different qualities on each side. More specifically, in some embodiments, the side of the fabric that touches the body is engineered to draw/absorb moisture from the body while the side of the fabric opposite the person is designed to wick and expel the moisture, and dissipate it away from the fabric. One such suitable fabric is available from Polartec. While the Polartec fabric is utilized in some embodiments, it is not to be construed as a requirement. In fact, other materials known in the art are fully contemplated herein. Water and air permeable materials will provide suitable cooling and heating while allowing moisture to pass away from the user's body. Thus, in some embodiments, where the packs described herein are to be used to cool a user, a surface of the body portion comprises a material that promotes heat and moisture removal from the user when worn by the user.

In embodiments, where the packs described herein are to be used to warm a user, a surface of the body portion comprises a material that inhibits heat removal from the user when worn by the user. For example, one or more portions of the pack (e.g., the surface contacting the user) may be made of a material designed and engineered to have warming properties. For example, fleece, Polartec, etc. or other warming fabrics may be used. Additionally, for warming purposes, the material could be thicker than for a pack used primarily for cooling. It is further noted that in some embodiments, since the thermoregulation material can be removably received in the receiving portions, the pack may be used for cooling and then used again for heating. In other words, the same pack may be used for both cooling and heating purposes in different uses through the use of different thermoregulation materials.

Furthermore, any of the packs described herein may include surfaces made of different materials. For example, the surface facing the user may be made of a different material than the surface facing away from the user.

Additionally, in some embodiments, at least a portion of the body portion of the packs described herein may be made of a material having a cooling or heating agent integrated into the material itself. For example, cooling or heating chemicals or fibers may be integrated, sewn, weaved or otherwise manufactured into the material of the pack. Examples of such fabrics specifically designed to have cooling properties include: ICE-FIL fabric commercially available from Ventex Co. Ltd., Seoul, Korea; DRIRELEASE fabrics commercially available from Optimer Performance Fibers, Wilmington, Del.; and COOLMAX fabrics commercially available from Invista of Wichita, Kans.

Further, in some embodiments, at least a portion of the body portion of the packs described herein may have a topical cooling or heating substance applied thereto. For example, a heating or cooling agent may be sprayed onto the surface of the pack that faces the user when worn, e.g., the surface that contacts or is in proximity to the user's skin. An example of an applicable cooling agent is Skins ICEBOOST commercially available from TriSports.com of Tucson, Ariz. Other examples could include cooling sprays including chemicals such as menthol, or heating sprays including chemicals such as capsaicin. Thus, generally, in some embodiments, other fabrics or materials could be used that offer cooling/heating properties via the construction of the fabric itself and/or via topical cooling sprays that may enhance the cooling/heating characteristics of the fabric or material.

In further embodiments, for the backing pad fabric, any of the thermoregulation packs described herein may utilize a technical performance fiber called Celliant™ made by Hologenix, LLC of Newport Beach, Calif. Celliant™ is a specially formulated fiber that is clinically proven to enhance oxygen levels in the body and help balance body temperature. In combination with the heating/cooling pack of some embodiments, a Celliant™-based thermoregulation pack is engineered to increase athletic performance and comfort. For example, in some embodiments, at least a portion of the pack contacting or in close proximity to the user's skin is made of a material including blood oxygen enhancing materials, such as a cloth or other material including Celliant™ fibers. In some embodiments, at least a surface of the breathable backing pad (e.g., as shown in FIG. 2, is made of such a blood oxygen enhancing material). Thus, in some embodiments, the body portion is made of a material having a blood oxygen enhancing substance integrated into the material.

An example of a use for the thermoregulation pack of some embodiments is for cyclists who want to regulate their temperature while riding, for example, in a race. In some embodiments, the thermoregulation pack (e.g., the body portion with the thermoregulation material and the straps) is made small enough in overall dimensions (e.g., thickness, but also including height and width in some cases) and weight to fit under a cyclist's shirt or jersey, but big enough to securely position the cooling/heating material proximate the user's skin to have the desired cooling effect. In some embodiments, an overall thickness of the thermoregulation pack is between about 1.5-3.0 cm, although it is understood that the dimensions will vary according to different applications and uses. Further, in some embodiments, the thermoregulation pack is made to fit under the cyclist's shirt without interfering with any other garment being worn, such as the cyclist's bib shorts. In some embodiments, the thermoregulation pack allows the cyclist to easily put on and take off the pack while riding. This is advantageous, for example, so that the cyclist may exchange the pack without stopping in the event the cooling pack loses effect. One advantage of wearing the thermoregulation pack under a cyclist's shirt is that sponsorship logos, images, designs, colors, text and/or information on the outside of the shirt are not covered by the pack. Another advantage of wearing the thermoregulation pack under a cyclist's shirt is that worn in conjunction with a tight fitting shirt or jersey, the pack contours to the shape of the cyclist's back and will not substantially increase aerodynamic resistance while the cyclist is riding.

Depending on the orientation of the user during use, the coupling mechanisms may or may not be needed. For example, for a cyclist who primarily rides in a forward leaning orientation, a coupling mechanism (such as a collar clip) may be sufficient to hold the pack in position since gravity will assist in holding the pack. However, for cyclists who ride in a more upright position, shoulder straps may be helpful to secure the pack in position. While reference is made to cyclists, the thermoregulation packs according to several embodiments may be worn by users in any number of athletic events, sports or activities or other casual, working, non-sport or other activities in which heating or cooling is needed to comfortably engage in such activities. In some embodiments, thermoregulation packs can be implemented without any need to modify other user equipment, clothing, uniform or jersey.

Furthermore, in accordance with one or more of the embodiments described herein, heating and cooling devices are provided in proximity to the skin of the user in order to facilitate the cooling and heating of a body shell, regulating the body shell temperature which will help regulate the core temperature of an athlete or user. In one or more embodiments, this may lead to increased performance or at least increased user comfort during the performance of activities or during physical exertion. Additionally, in some embodiments, the body portion is flexible (e.g., made of fabric) to assist in allowing the body portion to be held tightly against the spine of the user when worn.

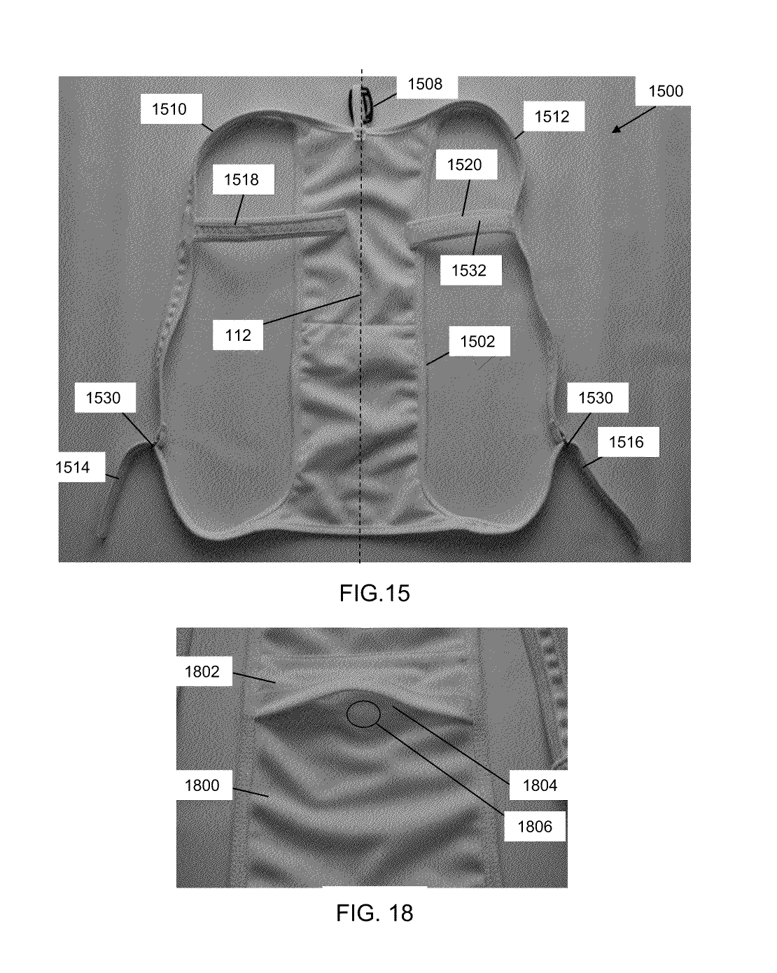

Referring next to FIGS. 15, 16 and 17, another embodiment of a thermoregulation pack 1500 (also referred to as a thermoregulation device) is shown. The pack 1500 includes a body portion 1502 having integrated straps 1510, 1512, 1514, 1520, chest straps 1518 and 1520, pouches 1522 and 1524 (generically referred to as receiving mechanisms) and a clip 1508 (generically referred to as a coupling mechanism). The pouches 1504 and 1506 each have a flap 1522 and 1524.

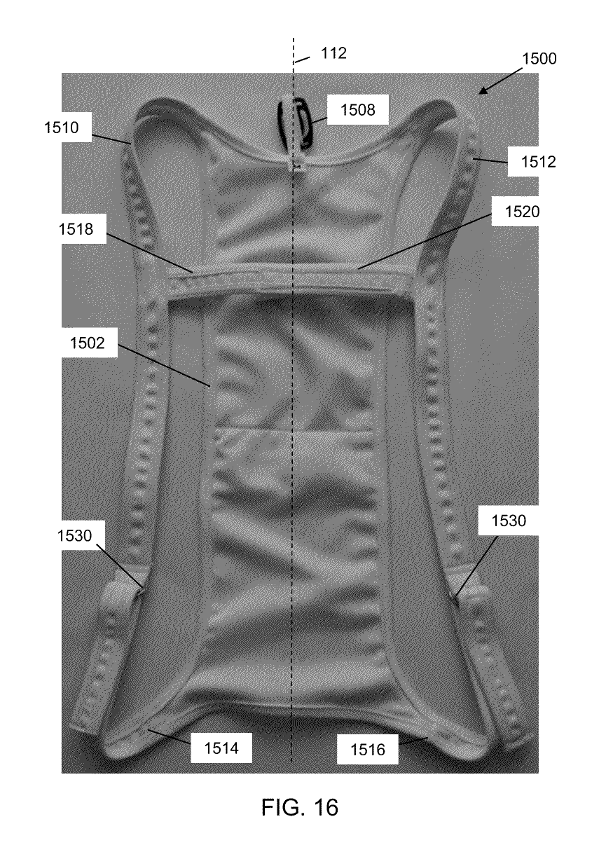

In this embodiment, the straps 1510, 1512, 1514, 1520, 1518 and 1520 are adjustable to assist in holding the body portion tightly against a spine of the user when worn such that the axis 112 is substantially aligned with the spine of the user. For example, straps 1510 and 1514 adjustably connect together, straps 1512 and 1516 adjustably connect together, and chest straps 1518 and 1520 adjustably connect together. In each case, a fabric hook and loop fastener is used to make the removable and adjustable connection (e.g., a VELCRO connection). As shown, the end of strap 1514 feeds through a ring 1530 at the end of strap 1510, then strap 1514 folds back and attaches to itself using a fabric hook and loop fastener. Similarly, the end of strap 1516 feeds through a ring 1530 at the end of strap 1512, then strap 1516 folds back and attaches to itself using a fabric hook and loop fastener. The use of the ring 1530 is known as a “looploc” connection. By feeding a variable amount of the straps 1514 and 1516 through the rings 1530, the pack tightness can be adjusted. FIG. 15 illustrates the straps 1514 and 1516 as fed through the rings 1530, folded back, but unconnected to themselves. FIG. 16 illustrates the straps 1514 and 1516 folded back and connected to themselves.

To help hold the pack in position, some embodiments include at least one horizontal strap, e.g., chest straps 1518 and 1520. In the illustrated embodiments, one end of each strap 1518, 1520 is integrated or fixed (e.g., sewn) to a portion of the upper straps 1510, 1512, and the other end of the straps 1518 and 1520 are adjustably and removably connectable to each other, e.g., using a fabric hook and loop fastener. FIG. 15 illustrates the chest straps 1518 and 1520 in the unconnected state and a fabric loop strip 1532 is visible. FIG. 16 illustrates the chest straps 1518 and 1520 connected together.

In some embodiments, the straps are configured to assist in holding the body portion 1502 tightly against a spine of the user when worn such that the axis 112 is substantially aligned with the spine of the user. Accordingly, the body portion 1502 and the pouches 1504 and 1506 are configured to hold the thermoregulation material in position along the axis 112 such that the thermoregulation material is held in proximity and adjacent to the spine of the user when worn by the user.

It is understood that the looploc and fabric hook and loop fastener connections may be replaced with any other removable connection devices known in the art. For example, in some case, ladder lock fasteners and/or buckle fasteners may be used.

It is understood that although a clip 1508 is illustrated, in some embodiments, a clip is not included such that the straps are sufficient to assist in holding the body portion 1502 tightly against a spine of the user when worn such that the axis 112 is substantially aligned with the spine of the user.

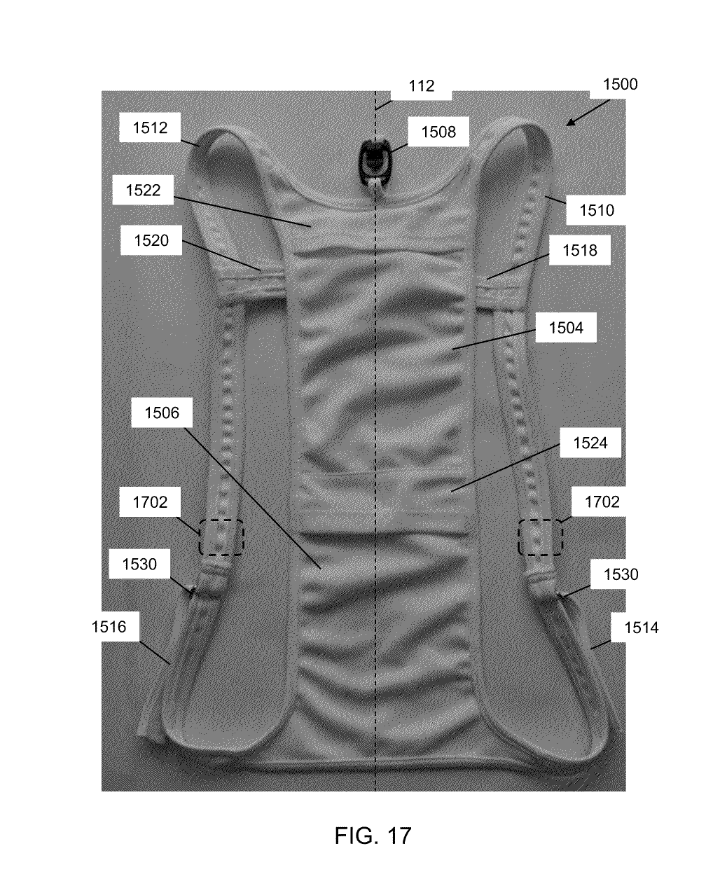

Additionally, it is noted that although chest straps 1518 and 1520 are shown, in some embodiments, abdominal straps are included, in addition to or instead of the chest straps. In one form, the abdominal straps would also be horizontal straps but attaching to the straps 1512 and 1514 at a location lower than the chest straps. For example, as shown in FIG. 17, the abdominal straps could attach at locations 1702 and then adjustably and removably connect to each other. In these embodiments, the abdominal straps assist in holding the lower portion of the body portion in place.

One or more of the straps described herein may be made of the same fabric as the remainder of the pack, or may include an adjustable connection device, and/or may be made of an elastic material.

It is also noted that FIG. 17 illustrates the two pouches 1504 and 1506 (without any thermoregulation material contained therein). The pouches are positioned along the axis 112 relative to each other.

Referring next to FIG. 18, shown is one embodiment of a flap 1802 of a pouch 1800 (one embodiment of a receiving portion) of a thermoregulation pack. In order to help secure the flap 1802 in position over the opening at an upper edge 1804 of the pouch 1800, a fastener 1806 is used. In one embodiment, the fastener 1806 is a fabric hook and loop fastener, for example, fastener 1806 is a fabric loop portion of the fabric hook and loop fastener, the hook portion attached to a corresponding undersurface of the flap 1802. It is understood that any other fasteners as described herein or otherwise understood in the art may be used.

Referring next to FIG. 19, views are shown of several variations of thermoregulation materials embodied as packs having multiple sections in multiple sizes in accordance with some embodiments, for use in correspondingly sized thermoregulation packs. Shown are thermoregulation materials embodied as packs 1902, 1904, 1906. Each pack has multiple sections 1908, 1910 and 1912. In some embodiments, each pack will insert into a given receiving portion of a given thermoregulation pack. The cooling/heating material or substance is divided between the multiple sections. Relative to a pack with a single section of cooling/heating material, the multiple sections spread or distribute the cooling/heating material. In particular with cooling packs, this can help to minimize slumping of the cooling material during thawing since the material even if thawed will stay in its section. It is noted that in cooling applications, the number of sections and size of section should be designed to provide the appropriate amount of cooling while minimize slumping of the thawing material. In smaller sections, cooling material will thaw more quickly (since there is less core cooling material). As illustrated, the sections 1908, 1910 and 1912 are positioned along the axis 112 relative to each other. This can be helpful to distribute the heating/cooling material along the axis 112 (which corresponds to the spine of the user). In some embodiments, the use of separations can provide added flexibility to the overall pack along the axis 112 since the pack will flex at the division between sections. It is noted that although each pack is shown with two sections, it is understood that the packs may be split into more than 2 sections.

Many embodiments are provided herein for thermoregulation packs or devices. One or more of the variations discussed in connection with any particular embodiment may be applicable to another embodiment. Generally, in one embodiment, a thermoregulation device to be worn by a user comprises a body portion having one or more receiving portions, the body portion having an axis extending about a height of the body portion, wherein the one or more receiving portions extend along the axis and each of the one or more receiving portions is configured to removably receive and hold in position a thermoregulation material; and at least one strap coupled to at least one portion of the body portion and configured to extend about a portion of the user when worn by the user. The at least one strap is configured to assist in holding the body portion tightly against a spine of the user when worn such that the axis is substantially aligned with the spine of the user. And the body portion and the one or more receiving portions are configured to hold the thermoregulation material in position along the axis such that the thermoregulation material is held in proximity and adjacent to the spine of the user when worn by the user.

In another embodiment, a thermoregulation device to be worn by a user, the thermoregulation device comprises: a body portion having one or more receiving portions, the body portion having an axis extending about a height of the body portion, wherein the one or more receiving portions extend along the axis and each of the one or more receiving portions is configured to removably receive and hold in position a thermoregulation material; and a coupling mechanism coupled to a first portion of the body portion and configured to removably couple the first portion of the body portion to a portion of a garment also worn by the user when the thermoregulation device is worn, wherein the coupling mechanism is configured to assist in holding the thermoregulation material in position along the axis such that the thermoregulation material is held in proximity and adjacent to the spine of the user when worn by the user.

In a further embodiment, a method of regulating a thermal characteristic of a user during physical exertion, the method comprises: inserting a thermoregulation material into one or more receiving portions of a body portion of a thermoregulation device, wherein the body portion has an axis extending about a height of the body portion, wherein the one or more receiving portions extend along the axis and each of the one or more receiving portions is configured to removably receive and hold in position the thermoregulation material along the axis; and positioning the body portion proximate to a back of the user such that the thermoregulation material is held in proximity and adjacent to a spine of the user; and using at least one strap coupled to at least one portion of the body portion to assist in holding the body portion tightly against the spine of the user such that the axis is substantially aligned with the spine of the user, wherein the at least one strap extends about a portion of the user. Additionally, in one variation, the method also includes removably coupling a first portion of the body portion to a portion of a garment worn by the user

While the invention herein disclosed has been described by means of specific embodiments, examples and applications thereof, numerous modifications and variations could be made thereto by those skilled in the art without departing from the scope of the invention set forth in the claims.

What is claimed is:

1. A thermoregulation device to be worn by a user, the thermoregulation device comprising:
a body portion having one or more receiving portions, the body portion having an axis extending about a height of the body portion, wherein the one or more receiving portions extend along the axis and each of the one or more receiving portions is configured to removably receive and hold in position a thermoregulation material; and
at least one strap coupled to at least one portion of the body portion and configured to extend about a portion of the user when worn by the user;
wherein the at least one strap is configured to assist in holding the body portion tightly against a spine of the user when worn such that the axis is substantially aligned with the spine of the user, and
wherein the body portion and the one or more receiving portions are configured to hold the thermoregulation material in position along the axis such that the thermoregulation material is held in proximity and adjacent to the spine of the user when worn by the user.

2. The thermoregulation device of claim 1 wherein the one or more receiving portions each comprises a pouch having an opening to removably receive the thermoregulation material such that the thermoregulation material is held within a volume of the pouch.

3. The thermoregulation device of claim 2 further comprising a flap covering the opening of the pouch.

4. The thermoregulation device of claim 1 further comprising an attachment mechanism to hold the flap in position over the opening of the pouch.

5. The thermoregulation device of claim 1 wherein the thermoregulation material comprises a heating or cooling pack containing a heating or cooling substance.

6. The thermoregulation device of claim 1 wherein the thermoregulation material comprises one or more packs, each pack having a plurality of sections, each section containing a thermoregulating substance.

7. The thermoregulation device of claim 6 wherein the plurality of sections are oriented along the axis relative to each other.

8. The thermoregulation device of claim 1 wherein the at least one strap is integral with the at least one portion of the body portion.

9. The thermoregulation device of claim 1 wherein the at least one strap is removably coupled to the at least one portion of the body portion, the body portion including at least one strap attachment mechanism to facilitate attachment and detachment of the at least one strap to the at least one portion of the body portion.

10. The thermoregulation device of claim 1 wherein the at least one strap comprises:
a first strap coupled to a first portion of the body portion; and
a second strap coupled to a second portion of the body portion, wherein the first strap and the second strap are removably connectable to each other.

11. The thermoregulation device of claim 10 wherein the first strap and the second strap are removably connectable to each other using a connection feature selected from a group consisting of: a fabric hook and loop fastener, a ladder lock fastener, a loop lock fasteners, a buckle fastener.

12. The thermoregulation device of claim 1 wherein the at least one strap comprises at least one shoulder strap.

13. The thermoregulation device of claim 1 wherein the at least one strap comprises at least one horizontal strap extending across a portion of a torso of the user when worn.

14. The thermoregulation device of claim 1 wherein the at least one strap comprises an elastic material.

15. The thermoregulation device of claim 1 further comprising a coupling mechanism coupled to a first portion of the body portion and configured to removably couple the first portion of the body portion to a portion of a garment worn by the user when the thermoregulation device is worn.

16. The thermoregulation device of claim 15 wherein the first portion of the body portion comprises an upper portion of the body portion, and the portion of the garment comprises a collar portion of the garment.

17. The thermoregulation device of claim 1 wherein the coupling mechanism comprises a clip.

18. The thermoregulation device of claim 1 wherein the body portion comprises a material that inhibits heat removal from the user when worn by the user.

19. The thermoregulation device of claim 1 wherein the body portion comprises a material that promotes heat and moisture removal from the user when worn by the user.

20. The thermoregulation device of claim 1 wherein the body portion comprises:
a first surface configured to face the user when worn; and
a second surface configured to face away from the user when worn,
wherein first surface and the second surface comprise different materials.

21. The thermoregulation device of claim 1 wherein the body portion is made of a material having a cooling or heating agent integrated into the material.

22. The thermoregulation device of claim 1 wherein the body portion is made of a material having a blood oxygen enhancing substance integrated into the material.

23. The thermoregulation device of claim 1 further comprising a cooling or heating substance applied to a surface of the body portion facing the user when worn.

24. The thermoregulation device of claim 1 wherein the body portion is flexible to assist in allowing the body portion to be held tightly against the spine of the user when worn.

25. The thermoregulation device of claim 1 wherein the body portion, the at least one strap and the thermoregulation material have dimensions and thicknesses such that the thermoregulation device can be worn under a fitted shirt also worn by the user.

26. A thermoregulation device to be worn by a user, the thermoregulation device comprising:
a body portion having one or more receiving portions, the body portion having an axis extending about a height of the body portion, wherein the one or more receiving portions extend along the axis and each of the one or more receiving portions is configured to removably receive and hold in position a thermoregulation material; and
a coupling mechanism coupled to a first portion of the body portion and configured to removably couple the first portion of the body portion to a portion of a garment also worn by the user when the thermoregulation device is worn, wherein the coupling mechanism is configured to assist in holding the thermoregulation material in position along the axis such that the thermoregulation material is held in proximity and adjacent to the spine of the user when worn by the user.

27. The thermoregulation device of claim 26 wherein the first portion of the body portion comprises an upper portion of the body portion, and the portion of the garment comprises a collar portion of the garment.

28. The thermoregulation device of claim 26 wherein the coupling mechanism comprises a clip.

29. A method of regulating a thermal characteristic of a user during physical exertion, the method comprising:
inserting a thermoregulation material into one or more receiving portions of a body portion of a thermoregulation device, wherein the body portion has an axis extending about a height of the body portion, wherein the one or more receiving portions extend along the axis and each of the one or more receiving portions is configured to removably receive and hold in position the thermoregulation material along the axis; and
positioning the body portion proximate to a back of the user such that the thermoregulation material is held in proximity and adjacent to a spine of the user; and
using at least one strap coupled to at least one portion of the body portion to assist in holding the body portion tightly against the spine of the user such that the axis is substantially aligned with the spine of the user, wherein the at least one strap extends about a portion of the user.

30. The method of claim 29 further comprising removably coupling a first portion of the body portion to a portion of a garment worn by the user.
